(19) United States
(12) Patent Application Publication
(10) Pub. No.: US 2024/0347146 A1
(43) Pub. Date: Oct. 17, 2024

(54) MULTINATIONAL CLINICAL DATA STANDARDIZATION METHOD AND DEVICE

(71) Applicant: MEDIAIPLUS, INC., Seongnam-si (KR)

(72) Inventors: Yong Jang JO, Seongnam-si (KR); Ji Hee JUNG, Seoul (KR)

(21) Appl. No.: 18/755,597

(22) Filed: Jun. 26, 2024

Related U.S. Application Data
(63) Continuation of application No. PCT/KR2021/020289, filed on Dec. 30, 2021.

(30) Foreign Application Priority Data
Dec. 28, 2021 (KR) 10-2021-0190379

(51) Int. Cl. G16H 10/20; G06F 16/25; G16H 50/70

(52) U.S. Cl. CPC G16H 10/20; G06F 16/258; G16H 50/70

(57) ABSTRACT

A multinational clinical data standardization method according to an embodiment may include the steps of: outputting individual entity names using a neural network model from multinational clinical data; refining the individual entity names; calculating similarities for the refined individual entity names; and standardizing the multinational clinical data by reflecting the similarity calculation result.

### TECHNICAL FIELD

The present disclosure relates to a multinational clinical data standardization method and device. More specifically, it relates to a multinational clinical data standardization method and device for a fast and easy access to multinational clinical data by priorly refining multinational clinical data and standardizing the data.

### BACKGROUND ART

A clinical trial is a trial involving human subjects in order to verify the safety, pharmacological effects and clinical effects of a medical substance. A clinical trial is an essential process in development of a medical substance because it is a procedure to secure safety of the medical substance and to confirm that the medical substance can go on sale. To prove the safety and efficacy of an investigational drug

Accordingly, analyzing and managing data of the past clinical trials is as important as planning clinical trials and proceeding with them. In order to conduct a clinical trial to be successful, mutual cooperation, among various organizations, entrusted organizations for clinical trials, and researchers, is essential. However, it is not easy to find entrusted organizations for clinical trials or researchers specializing in various diseases or medicines. In addition, since, even in case of a same hospital, an entrusted organization, or a researcher, they are described in different methods or in different languages, it is very challenging to exactly identifying them.

As the society has been transformed into a knowledge-based society, knowledge as a means of production came into the spotlight, and corporations began establishing and managing a knowledge management system (KMS) for systematically managing knowledge scattered within the corporations. However, the existing knowledge management system (KMS) cannot reflect characteristics of clinical data, such as the need of handling importantly the latest clinical data, in particular, or the need of searching for clinical data that has been registered only in other countries to be difficult for local searches.

### SUMMARY

In order to solve the aforementioned problem, the present disclosure is aimed at providing a method for efficiently searching for clinical data that a user wants, by converting clinical data expressed in different methods into standardized data.

In particular, the present disclosure is aimed at providing a method for conducting a more exact and rapid standardization by processing and classifying data so that previously refined data can be used for the standardization.

According to an embodiment the present disclosure, a multinational clinical data standardization method may include the steps of: outputting names of individual entities from multinational clinical data using a neural network model; refining the names of individual entities; calculating similarities of the refined names of the individual entities; and performing standardization of the multinational clinical data by reflecting results of the similarity calculation.

The step of refining the names may include a step of, when names of at least two individual entities correspond to one attribute and if a predetermined criterion is satisfied, separating the names of the at least two individual entities such that they correspond respectively to two attributes.

The step of refining the names may include a step of, when names of at least two individual entities correspond to at least two attributes and if a predetermined criterion is satisfied, merging the names of the at least two individual entities such that they correspond to one attribute.

The step of calculating similarities may include the steps of acquiring sets of character strings corresponding to the refined names of individual entities; calculating a distance value between two of the sets of character strings; and calculating a similarity based on the calculated distance value.

The step of calculating a distance value may include calculating the distance value based on the number of characters inserted into a second character string forming a set of second character strings, the number of characters removed from it, and the number of characters replaced within it, by using as a reference a first character string forming a set of first character strings among the two sets of character strings.

In the step of calculating similarities, the distance value may be calculated by giving low weightings on the number of inserted characters and the number of removed characters and giving a high weighting on the number of the replaced characters.

The step of performing standardization may include the steps of arranging names of individual entities corresponding to the two sets of character strings, having similarities equal to or higher than a predetermined threshold value, to be one name; and performing standardization of the multinational clinical data by reflecting results from the aforementioned step of arrangement.

The method may further include the step of converting multinational clinical data written in a hierarchical database (DB) format into data in a relational DB format. In the step of outputting names of individual entities, the names of individual entities may be outputted from the multinational clinical data written in the relational DB format by using a neural network model.

According to an embodiment the present disclosure, a multinational clinical data standardization device may comprise a memory to store multinational clinical data; and a processor to output names of individual entities from multinational clinical data using a neural network model, to refine the names of individual entities, to calculate similarities of the refined names of the individual entities, and to standardize the multinational clinical data by reflecting results of the similarity calculation.

The present disclosure allows efficiently searching for clinical trial data that a user wants, by converting clinical trial data expressed in different methods into standardized data.

In particular, the present disclosure allows conducting a more exact and rapid standardization by processing and classifying data so that previously refined data can be used for the standardization.

### BRIEF DESCRIPTION OF DRAWINGS

FIG. 1 is a block diagram of a multinational clinical data standardization device (1) according to an embodiment.

FIG. 2 is a flow diagram illustrating a multinational clinical data standardization method according to an embodiment;

FIG. 6 and FIG. 7 are flow diagrams specifically illustrating s3 in FIG. 2;

FIG. 10 is a flow diagram specifically illustrating s4 in FIG. 2; and

FIG. 3, FIG. 4, FIG. 5, FIG. 8, FIG. 9, FIG. 11, and FIG. 12 are diagrams referred to when illustrating the aforementioned flow diagrams.

### DETAILED DESCRIPTION OF EXEMPLARY EMBODIMENTS

In the following detailed description, reference is made to the accompanying drawings that show, by way of illustration, specific embodiments in which the present invention may be implemented. These embodiments are described in sufficient detail to enable those skilled in the art to implement the present invention. It is to be understood that the various embodiments of the present invention, although different, are not necessarily mutually exclusive. For example, a particular feature, structure or characteristic described herein in connection with one embodiment may be implemented within other embodiments without departing from the spirit and scope of the present invention. In addition, it is to be understood that the location or arrangement of individual elements within each disclosed embodiment may be modified without departing from the spirit and scope of the present invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar elements throughout the several views.

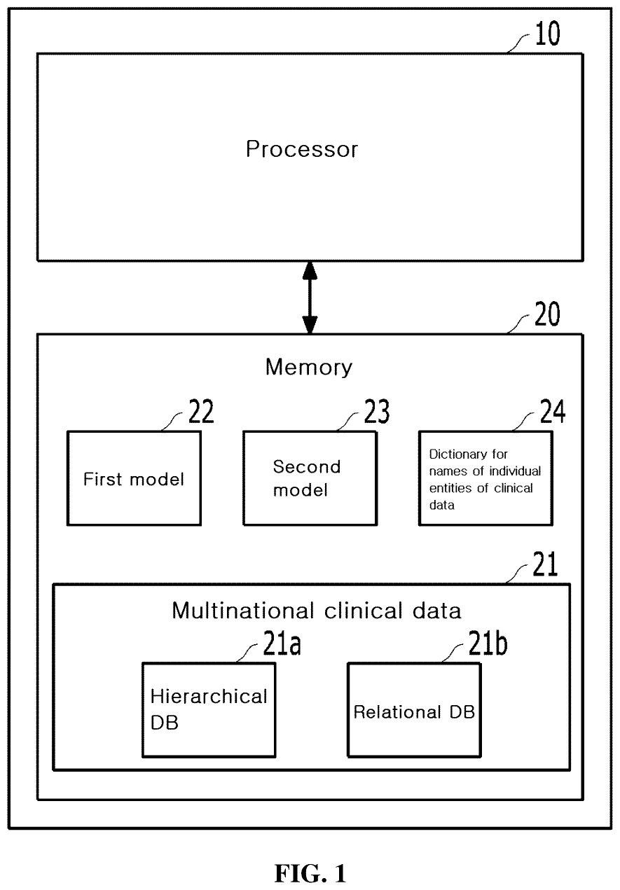

FIG. 1 is a block diagram of a multinational clinical data standardization device 1 according to an embodiment.

A multinational clinical data standardization device 1 according to an embodiment may comprise a processor 10 and a memory 20.

The memory 20 may store various programs and data required for operations of the multinational clinical data standardization device 1. The memory 20 may store a multinational clinical data database (DB) 21, a first model 22, a second model 23, and a dictionary for names of individual entities of clinical data 24.

The processor 10 may store clinical data, collected from a home country and other various countries, in the multinational clinical data DB 21. The multinational clinical data DB 21 may contain at least one of titles of clinical trials; names of organizations; names of diseases; names of medicines; information of researchers; genders, ages, names of subjects; methods of trials. The multinational clinical data DB21 may be formed of a hierarchical DB 21a and a relational DB 21b.

The multinational clinical data DB 21 may store clinical data for every clinical trial in a form of a document and by assigning a unique identification code to each clinical trial.

As shown in FIG. 3, the hierarchical DB 21a may be a DB having a hierarchical structure in which a record is stored in a segment having a hierarchical structure. The hierarchical DB 21a may have a structure formed with parent segments and child segments connected with each other. In the hierarchical DB 21a, one parent segment may be connected with at least one child segment. A segment may be a node of the hierarchical DB 21a mapped with a table of the relational DB 21b. The hierarchical DB 21a and the relational DB 21b may have a relation in which records are mapped with each other.

The relational DB 21b may be an assembly in which tables get together, wherein each table, formed of rows and columns, establishes relations with other tables. A row may be a tuple and/or a record. A column may be a field and/or an attribute. Mapping means matching records present in tables of the relational DB 21b with records present in segments of the hierarchical DB 21a.

A first model 22 may be a model implemented to output names of individual entities from the multinational clinical data by the processor 10.

The first model 22 may be acquired by the processor 10 through a neural network learning for obtaining individual entity names included in the multinational clinical data for learning based on multinational clinical data for learning.

According to an embodiment, a Named Entity Recognition (NER) model may be used as the first model 22. Named entity recognition, which means recognizing entities, each with a specific name, may be implemented by an algorithm to recognize a type to which a word meaning a certain name belongs.

A second model 23 may be achieved by applying embeddings reflecting contexts in the multinational clinical data by the processor 10. An embedding of words/sentences/entities/documents reflecting contexts is a method of expressing the words/sentences/entities/documents in a low-dimensional space. Since this is to embed words/sentences/entities/documents differently depending on contexts even the words/sentences/entities/documents have the same writings, different vector values may be extracted from the words/sentences/entities/documents having the same writings depending on the contexts.

The second model 23 may be acquired by the processor 10 through a neural network learning for obtaining context-based embedding values for the multinational clinical data for learning based on multinational clinical data for learning. Specifically, the second model 23 may be acquired by the processor 10 through learnings, each for obtaining a context-based embedding value for each word/sentence/entity/document of the multinational clinical data for learning.

According to an embodiment, a Bidirectional Encoder Representations from Transformers (BERT) model may be used as the second model 23. The BERT model, which is a natural language processing (NLP) model to bidirectionally learn sentences, is established by performing a pre-learning using words of dictionaries already registered and a fine-tuning of the learned model. Through the fine-tuning process, the BERT model has high accuracy even when the data amount is small. Further, since it is an attention-based model to enhance the performance by making a specific vector focused, even when a sentence is long, its performance is not decreased and the accuracy may be maintained. However, the BERT model is merely an example. Any models, which are able to extract context-based vector values, may be applied to the present disclosure.

The processor 10 may control overall operations of the multinational clinical data standardization device 1.

Specifically, the processor 10 may output names of individual entities from multinational clinical data by using a neural network model, refine the names of individual entities, calculate similarities of the refined names of individual entities, and standardize the multinational clinical data by reflecting results of the similarity calculation,.

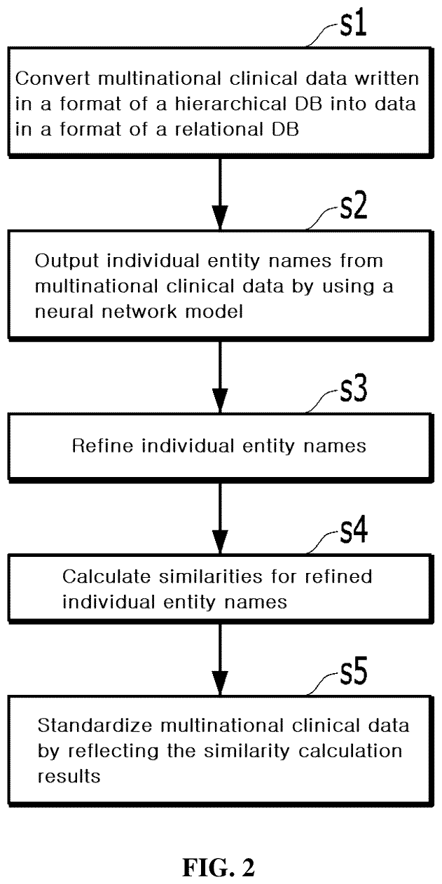

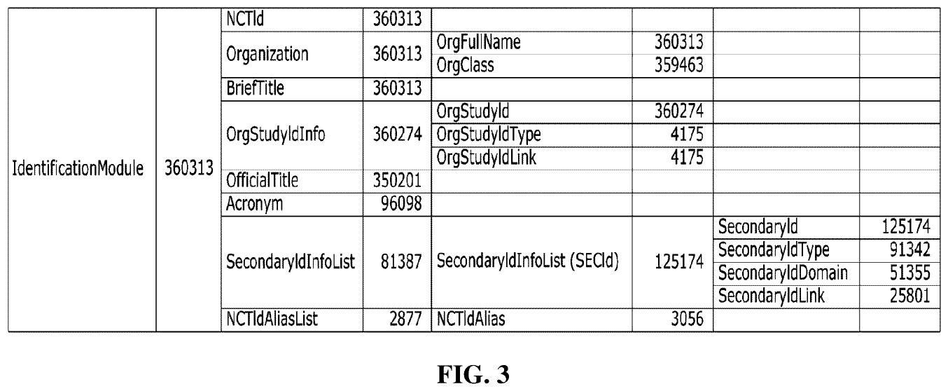

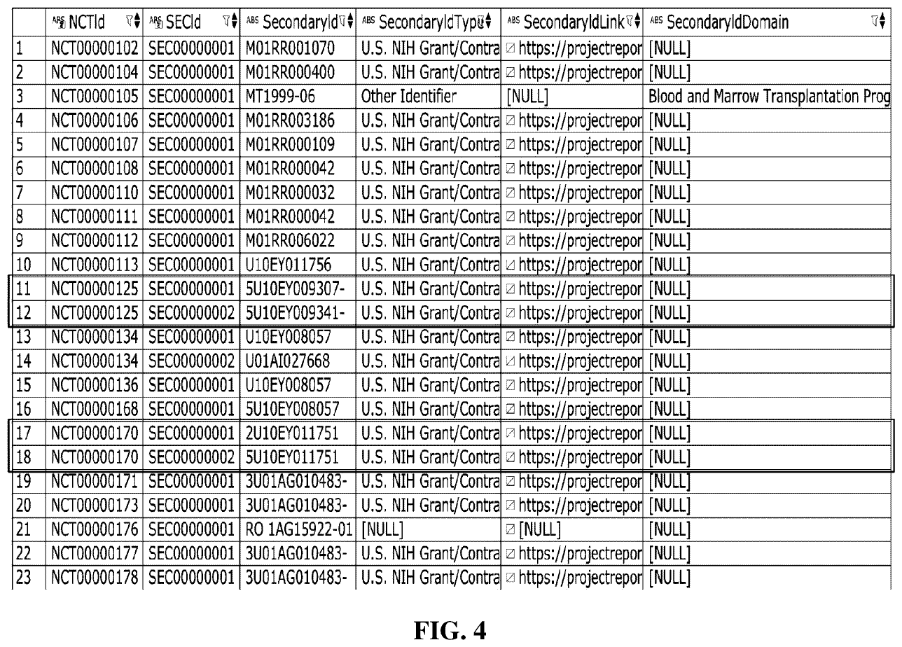

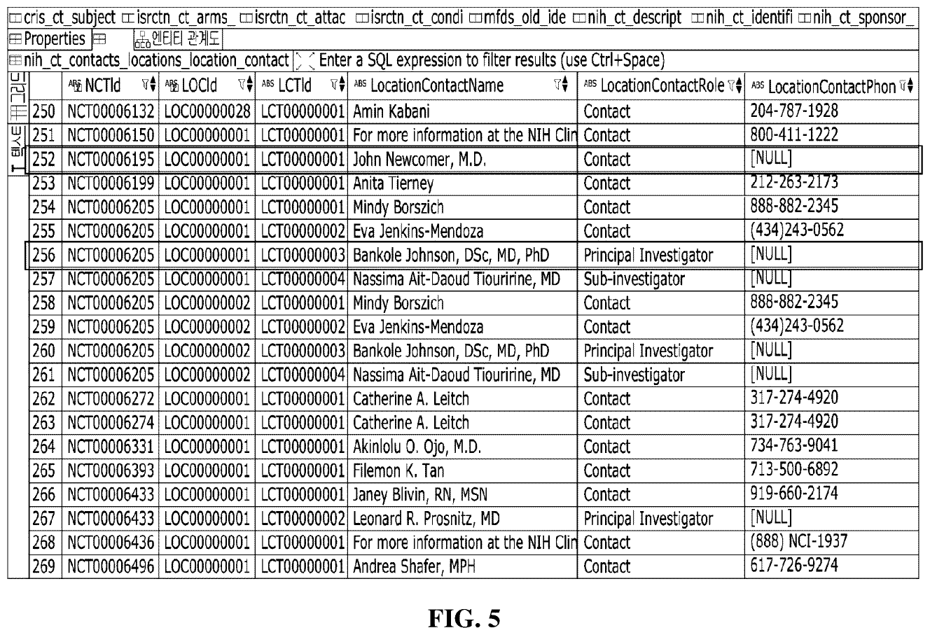

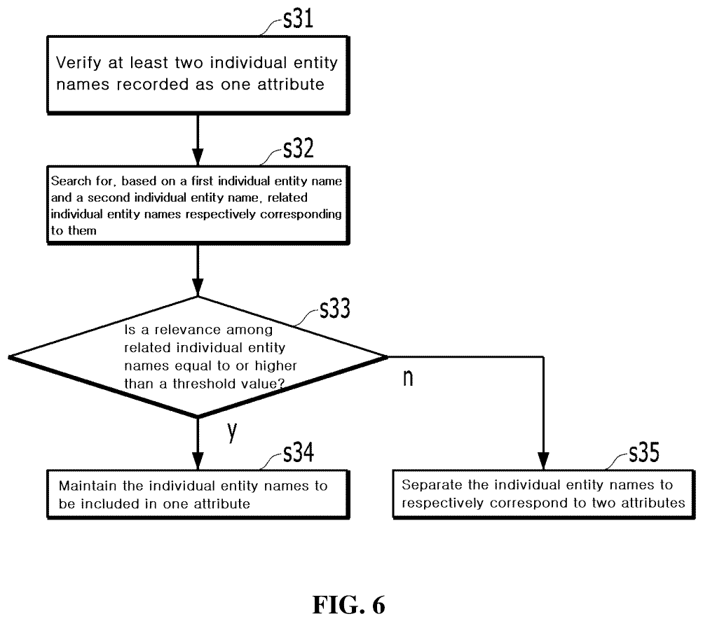

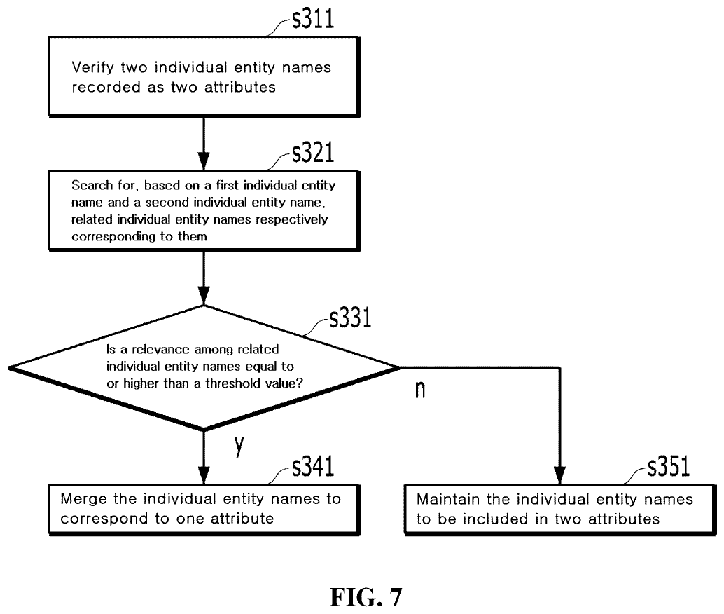

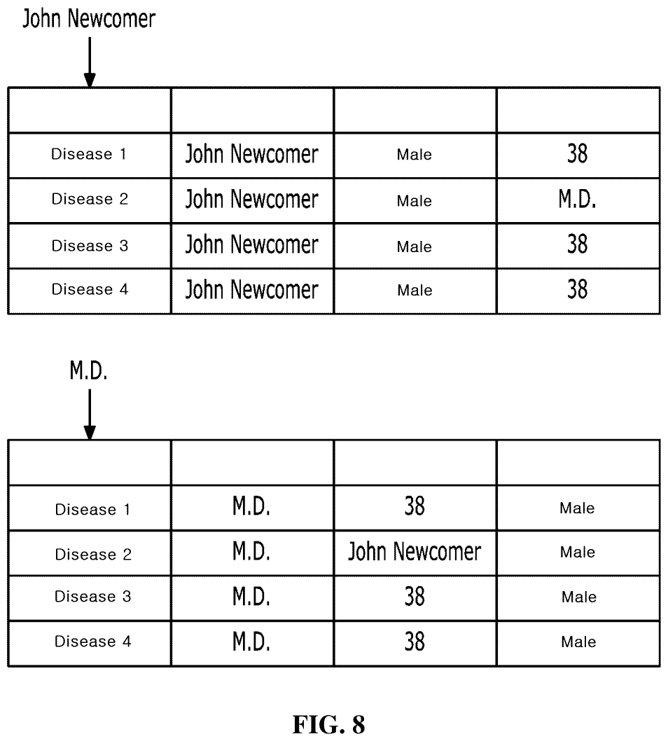

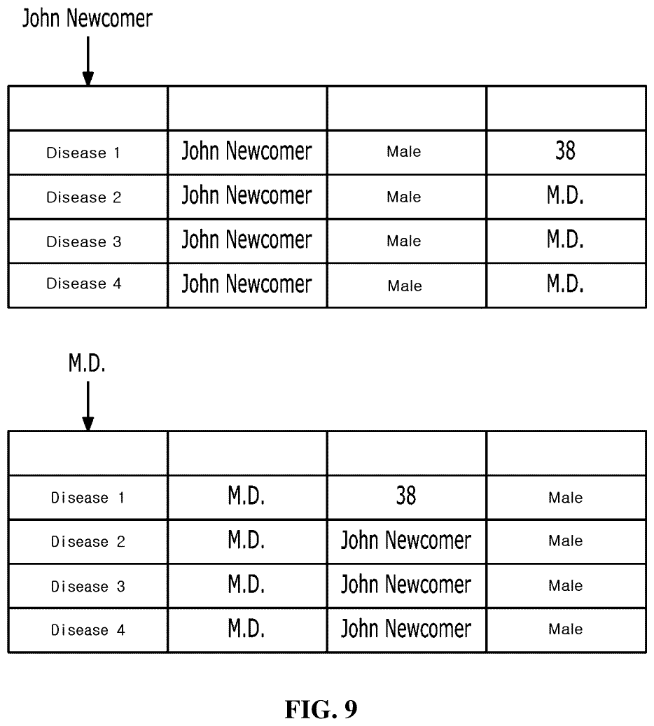

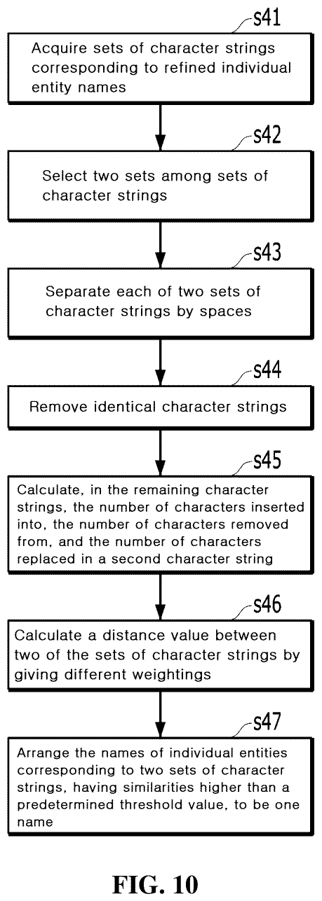

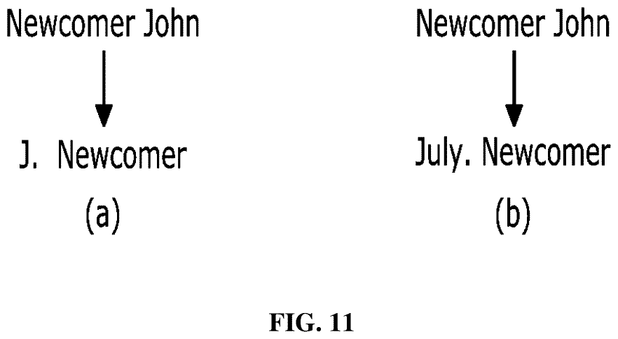

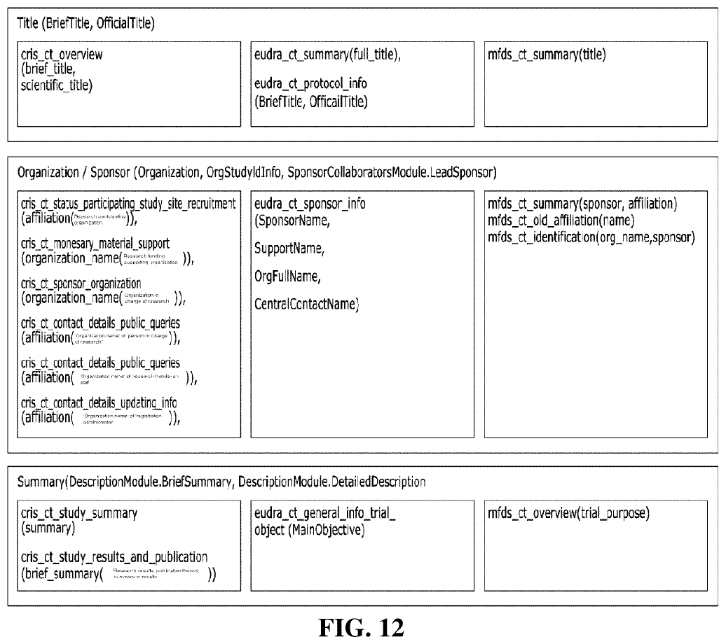

FIG. 2 is a flow diagram illustrating a multinational clinical data standardization method according to an embodiment; FIG. 6 and FIG. 7 are flow diagrams specifically illustrating s3 in FIG. 2; FIG. 10 is a flow diagram specifically illustrating s4 in FIG. 2; and FIG. 3 to FIG. 5, FIG. 8, FIG. 9, FIG. 11, and FIG. 12 are diagrams referred to when illustrating the aforementioned flow diagrams.

Hereinafter, the descriptions will be made with reference to FIG. 2 to FIG. 12. For reference, each step of each flow diagram is merely an example. Even in a case when the sequences of the steps are differently modified and/or combined, the present disclosure may be identically/similarly applied.

According to an embodiment, the processor 10 may convert multinational clinical data written in a format of a hierarchical DB 21a into data in a format of relational DB 21b (s1).

According to an embodiment, not only in a case when the number of pieces of data of one child attribute is the same as that of one parent attribute to which the child attribute corresponds, but also in a case when the number of pieces of data of one child attribute is different from (that is, greater than) that of one parent attribute to which the child attribute corresponds, the processor 10 may convert multinational clinical data written in a format of a hierarchical DB 21a into data in a format of a relational DB 21b.

For example, as shown in FIG. 3, not only in a case when the number of pieces of data of a child attribute ‘OrgFullNam’ is identical to that of a parent attribute ‘Organization’, to which the child attribute corresponds, as 340313, but also in a case when the number (125174) of pieces of data of a child attribute ‘Secondary Info (SECid)’ is greater than the number (81387) of pieces of data of a parent attribute ‘SecondaryInfoList’, to which the child attribute corresponds, the multinational clinical data may be converted into data in a format of a relational DB 21b as shown in FIG. 4.

That is, the latter case means that identical attributes are repeated. According to an embodiment, even in a case of a hierarchical DB 21a in such a type, the data may be converted into data in a format of relational DB 21b by re-defining relations between attributes or adding new attributes. In this way, even when a hierarchical DB 21a has a complicated data structure in which identical attributes are repeated, it may be easily converted into a relational DB 21b.

The processor 10 may output names of individual entities from multinational clinical data written in a format of a relational DB 21b by using the first model 22 (s2).

Specifically, the processor 10 may output names of individual entities from the multinational clinical data by using the first model 22 and output embedding values for respective individual entity names reflecting contexts of the respective individual entity names by using the second model 23.

Specifically, the processor 10 may perform recognition of individual entity names by using the first model 22. The processor 10 may perform recognition of individual entity names of the multinational clinical data by using the dictionary for names of individual entities 24 stored in the memory 20. For example, names of diseases, entrusted organizations for clinical trials, symptoms, remedial agents, conditions for joining clinical trials, or the like may be recognized. The dictionary for names of individual entities 24 may contain a plurality of individual entity names corresponding to the multinational clinical data and a plurality of synonyms corresponding to the respective individual entity names.

According to an embodiment, the processor 10 may determine whether the recognition of individual entity names is successfully performed by determining whether an individual entity name recognized by the recognition of individual entity names is included in the dictionary for names of individual entities 24. When the individual entity name is included in the dictionary for names of individual entities 24, it may be determined to be successful in the NER. On the contrary, if the individual entity name is not included in the dictionary for names of individual entities 24, it may be determined to be failed in the NER.

According to an embodiment, the processor 10 may perform a Part-of-Speech (POS) tagging with respect to the names of individual entities outputted by the first model 22. That is, parts of speech, such as nouns, adjectives, verbs, or the like may be marked for individual entity names.

According to an embodiment, the processor 10 may output embedding values for respective individual entity names reflecting contexts of the respective individual entity names by using the second model 23 for the names of individual entities outputted by using the first model 22 and/or for which the POS tagging has been performed. The processor 10 may create a token for each individual entity name and input the tokenized individual entity name into the second model 23 to output an embedding value for each individual entity name. The processor 10 may output embedding values for individual entity names for each clinical trial document.

According to the present disclosure, result information, from the performance of the POS tagging for the individual entity names and/or the context embedding, may be used later when calculating similarities of individual entity names (s4). In this way, more accurate similarity determination regarding the multinational clinical data may be conducted. Descriptions in this regard will be made below with reference to FIG. 4.

The processor 10 may perform refinement of the names of individual entities (s3).

Referring to record 252 and record 256 in FIG. 5 as an example, it is noticed that each of the two records includes two or more names of individual entities (John Newcomer, M.D./Bankole Johnson, Dsc, MD, PhD) under one attribute (LocationContactName). For this attribute, only a person's name is required and names of its profession (or its position), such as MD or PhD, are not needed.

According to an embodiment, in such a case where at least two names of individual entities correspond to one attribute, the processor 10 may separate the names of individual entities to correspond to at least two attributes based on a predetermined criterion.

Specifically, referring to FIG. 6 and FIG. 8, the processor 10 may verify at least two names of individual entities recorded as one attribute (s31). According to an embodiment, it can be verified whether there are at least two names of individual entities recorded as one attribute in one record.

Then, the processor 10 may search for, based on a first individual entity name among at least two individual entity names, related individual entity names corresponding to the first individual entity name in the multinational clinical data DB 21 (s32). Specifically, the processor 10 may receive a question for search with a first individual entity name as a keyword inputted by a user and search for related individual entity names (for example, male, 38, M.D.) corresponding to a first individual entity name (for example, John Newcomer) in the multinational clinical data DB 21.

Likewise, the processor 10 may search for, based on a second individual entity name among at least two individual entity names, related individual entity names corresponding to the second individual entity name in the multinational clinical data DB21. Specifically, the processor 10 may receive a question for search with a second individual entity name as a keyword inputted by a user and search for related individual entity names (for example, male, 38, John Newcomer) corresponding to a second individual entity name (for example, M.D.) in the multinational clinical data DB 21.

For reference, FIG. 8 shows, as an example, lists of related individual entity names using names of diseases as a criterion. To lists using as criterions other attributes, such as names of organizations, the present disclosure may also be applied identically/similarly.

The processor 10 may determine whether a relevance among related individual entity names is equal to or higher than a threshold value (s33). Specifically, the processor 10 may determine whether a relevance, between related individual entity names in a first list of disease names acquired based on the first individual entity name and related individual entity names in a second list of disease names acquired based on the second individual entity name, is equal to or higher than a threshold value. More specifically, a relevance may be determined based on a ratio of the second individual entity name included in the first list of disease names and a ratio of the first individual entity name included in the second list of disease names.

When the relevance is determined to be equal to or higher than the threshold value, it is determined to maintain the first individual entity name and the second individual entity name to be included in one attribute (s34). On the contrary, when the relevance is determined to be lower than the threshold value, the first individual entity name and the second individual entity name may be separated to be included respectively in two attributes (s35). Referring to FIG. 8, for example, since the ratio of the appearance of the name ‘M.D.’ among the related individual entity names corresponding to the name ‘John Newcomer’ is lower than the threshold value, and the ratio of the appearance of the name ‘John Newcomer’ among the related individual entity names corresponding to the name ‘M.D.’ is lower than the threshold value, the separation of these individual entity names may be conducted.

Referring to FIG. 7 and FIG. 9, the processor 10 may verify at least two individual entity names recorded as at least two attributes (s311). According to an embodiment, it can be verified whether there are at least two names of individual entities recorded as at least two attributes in one record.

The processor 10 may search for, based on a first individual entity name among at least two individual entity names, related individual entity names corresponding to the first individual entity name in the multinational clinical data DB 21, and search for, based on a second individual entity name among at least two individual entity names, related individual entity names corresponding to the second individual entity name in the multinational clinical data DB 21 (s321). Then, the processor 10 may determine whether a relevance among related individual entity names is equal to or higher than a threshold value (s331). Steps s321 and s331 of FIG. 7 may be identically/similarly applied to steps s32 and s33 of FIG. 6.

When the relevance is determined to be equal to or higher than the threshold value, the first individual entity name and the second individual entity name may be merged to be included in one attribute (s341). On the contrary, when the relevance is determined to be lower than the threshold value, the first individual entity name and the second individual entity name may be maintained to correspond to two attributes (s351). For example, referring to FIG. 9, since the ratio of the appearance of the individual entity name ‘M.D.’ among the related individual entity names corresponding to the individual entity name ‘John Newcomer’ is equal to or higher than the threshold value, and the ratio of the appearance of the individual entity name ‘John Newcomer’ among the related individual entity names corresponding to the individual entity name ‘M.D.’ is equal to or higher than the threshold value, the merger of these individual entity names may be conducted.

That is, a database from which noise is removed, in other words, a more refined database may be acquired by determining the relevance among keywords, based on the appearance ratio of a target keyword in searches of respective keywords, and determining based on the relevance whether to maintain the keywords to be included in one attribute. Since the standardization is conducted based on the refined data, the standardization of unnecessary data will not be conducted, resulting in enhancing the data processing speed.

The processor 10 may calculate similarities for the refined named of individual entities (s4).

Specifically, referring to FIG. 10 and FIG. 11, the processor 10 may acquire sets of character strings corresponding to the refined individual entity names (s41) and select two sets among the sets of character strings (s42).

According to an embodiment, the processor 10 may select two sets of character strings based on results from false similarity determinations for names of individual entities.

According to an embodiment, in a state where the POS tagging and/or the context embedding have already been conducted for the individual entity names, the processor 10 may determine false similarities for names of individual entities based on information resulting from the conduct of them. Then, the processor 10 may compare embedding values for respective individual entity names and select sets of character strings for names of individual entities having false similarities equal to or higher than a threshold value. For example, the sets of character strings ‘Newcomer John’ and ‘J. Newcomer’ may be selected.

The processor 10 may calculate a distance value between two sets of character strings.

Specifically, character strings in each of the two sets may be separated by spaces (s43). Then, the separated character strings may be compared with each other and identical character strings are removed (s44). The processor 10 may calculate the number of characters inserted into a second character string compared with a first character string, the number of characters removed from it, and the number of characters replaced in it (s45).

For example, referring to the character strings of (a) ‘Newcomer John’ and ‘J. Newcomer’, the identical character strings ‘Newcomer’ may be removed and the character strings ‘John’ and ‘J’ may be compared to calculate the number of removed characters, which are ‘o’, ‘h’, and ‘n’, totaling 3. Referring to the character strings of (b) ‘Newcomer John’ and ‘July. Newcomer’, the identical character strings ‘Newcomer’ may be removed and the character strings ‘John’ and ‘July’ may be compared to calculate the number of replaced characters, which are ‘u’, ‘l’, and ‘y’, totaling 3.

The processor 10 may give different weightings to the number of inserted characters (or the number of removed characters) and the number of replaced characters to calculate a distance value between the two sets of character strings (s46). According to an embodiment, the distance value may be calculated by giving relatively low weightings to the number of inserted characters and the number of removed characters and giving a relatively high weighting to the number of replaced characters. Here, to the number of inserted characters and the number of removed characters, the same weighting may be given.

For example, when giving a weighting of 0.1 to the number of inserted characters and the number of removed characters and giving a weighting of 1 to the number of replaced characters, 0.3 of a distance value is calculated in case of (a) and 3 of a distance value is calculated in case of (b).

The processor 10 may calculate similarities among the refined names of individual entities based on the calculated distance values. Specifically, the processor 10 may determine that the individual entity names are similar when a distance value is less than a predetermined threshold value. In particular, when a similarity is high, it may be determined to be identical. On the contrary, when a distance value is equal to or higher than a predetermined threshold value, the relevant names of individual entities may be determined to be different.

For example, the names of individual entities in (a) may be determined to be identical and the names of individual entities in (b) may be determined to be different.

Since it is highly likely that a same name is written differently (for example, in abbreviation) when some characters are inserted or removed, a low weighting is assigned. However, when some characters are replaced, it is highly likely that the names are recognized to be different, and therefore, a high weighting is assigned.

According to an embodiment, the examples in FIG. 10 and FIG. 11 may be usefully applied in particular when an individual entity name is a type of abbreviation. For example, in a case when there are possibilities that organization names, person names, disease names, or the like are written in abbreviations, it may be prevented that names of individual entities in abbreviations are recognized to be different from original names of individual entities, thereby constructing a database containing more exact contents.

The processor 10 may deem individual entity names, corresponding to two sets of characters strings with a similarity equal to or higher than a threshold value, as one individual entity name and arrange them in the database (s47).

That is, the processor 10 may determine that the names of individual entities ‘Newcomer John’ and ‘J. Newcomer’ to be identical ones, select one of these two as a representative individual entity name, change the rest into the representative individual entity name, and arrange them in the database.

Although FIG. 10 and FIG. 11 show an example that two sets of character strings are selected, the present disclosure would also be identically/similarly applied to a case where at least two sets of character strings are selected and compared.

The processor 10 may standardize the multinational clinical data by reflecting results of the similarity calculation for the refined names of individual entities (s5).

Specifically, in the multinational clinical data DB 21, several names of each attribute different depending on countries and/or organizations may be classified under one unified name. The classified data may be converted to have standards optimized for searches. For example, referring to FIG. 12, the attribute ‘cris_vt_overview’ regarding ‘CRIS’, the attribute ‘eudra_ct_summary’ regarding ‘eudra’, and the attribute ‘mfds_ct_summary’ regarding ‘mfds’ may be classified under one attribute called ‘Title (BriefTitle, OfficialTitle)’.

The standardization may include lowercasing English characters included in the data or removing adjectives, adverbs, prepositions, and special characters. Here, additional dictionaries for stopwords may be utilized and, if necessary, spell-checks may be performed so that typographical errors or incorrectly written terms may be converted into standard terms.

The standardization may include unifying languages, such as converting data in foreign languages into data in a language of a home country or converting data in a language of a home country into data in a foreign language; processing data mainly based on terms related to clinical trials and frequently used in the field of clinical trials; or unifying terms having identical meanings or interpreted into similar meanings into one unified term. Here, unified terms may be terms used by persons skilled in the art in the field of clinical trials. In addition, the standardization may further include converting terms, that are not used any more in the field of clinical trials, into replaced terms that are currently used in the field of clinical trials.

According to the present disclosure, the standardization is conducted after refining names of individual entities in step s3 and calculating similarities of the refined names of individual entities in step s4 to arrange the names of individual entities to be unified names. In this way, when standardizing massive multinational clinical data, the processing speed may be enhanced.

The embodiments described above can be implemented in a form of an executable program command through a variety of computer means recordable to computer readable media. The computer readable media may include solely or in combination, program commands, data files, and data structures.

The program commands recorded to the media may be components specially designed for the present invention or may be usable by a skilled person in a field of computer software.

Computer readable recording media include magnetic media such as a hard disk, a floppy disk, magnetic tape, an optical media such as a CD-ROM and a DVD, a magneto-optical media such as a floptical disk and hardware devices such as ROM, RAM, and flash memory specially designed to store and carry out programs. Program commands include not only a machine language code made by a complier, but also a high level code that can be used by an interpreter etc., which is executed by a computer. The aforementioned hardware device can work as more than a software module to perform the action of the present invention, and they can do the same in the opposite case.

Aspects of the present disclosure may take a form of hardware overall, software (including firmware, resident software, micro codes, or the like) overall, or computer program products embodied in at least one computer readable medium on which computer readable program codes are implemented.

Characteristics, structures, effects, etc. described above regarding the embodiments are included in one embodiment of the present disclosure, but not necessarily limited to one embodiment. Further, characteristics, structure, effects, etc. exemplarily illustrated in each embodiment may be implemented regarding other embodiments through combinations or modifications by persons skilled in the art in the field to which the embodiments belong. Accordingly, contents associated to such combinations or modifications should be interpreted to be included in the scope of the present disclosure.

In addition, although the present disclosure is described with respect to some embodiments in this specification, these embodiments are merely examples and do not limit the present disclosure. Those skilled in the art will know that various modifications and applications, not illustrated above, are possible within the scope without departing from the essential characteristics of the present disclosure. For example, each component specifically described in the embodiments may be modified. Differences related to such modifications and applications should be interpreted to be included in the scope of the present disclosure that the accompanying claims prescribe.

### INDUSTRIAL APPLICABILITY

The present disclosure allows efficiently searching for clinical trial data that a user wants, by converting clinical trial data expressed in different methods into standardized data.

In particular, the present disclosure allows conducting a more exact and rapid standardization by processing and classifying data so that previously refined data can be used for the standardization.

1. A multinational clinical data standardization method including the steps of:
outputting names of individual entities from multinational clinical data using a neural network model;
refining the names of individual entities;
calculating similarities of the refined names of the individual entities; and
performing standardization of the multinational clinical data by reflecting results of the similarity calculation.

2. The multinational clinical data standardization method of claim 1, wherein the step of refining the names includes a step of, when names of at least two individual entities correspond to one attribute and if a predetermined criterion is satisfied, separating the names of the at least two individual entities such that they correspond respectively to two attributes.

3. The multinational clinical data standardization method of claim 1, wherein the step of refining the names includes a step of, when names of at least two individual entities correspond to at least two attributes and if a predetermined criterion is satisfied, merging the names of the at least two individual entities such that they correspond to one attribute.

4. The multinational clinical data standardization method of claim 1, wherein the step of calculating similarities includes the steps of acquiring sets of character strings corresponding to the refined names of individual entities; calculating a distance value between two of the sets of character strings; and calculating a similarity based on the calculated distance value.

5. The multinational clinical data standardization method of claim 4, wherein the step of calculating a distance value includes calculating the distance value based on the number of characters inserted into a second character string forming a set of second character strings, the number of characters removed from it, and the number of characters replaced in it, by using as a reference a first character string forming a set of first character strings among the two sets of character strings.

6. The multinational clinical data standardization method of claim 5, wherein, in the step of calculating similarities, the distance value is calculated by giving low weightings on the number of inserted characters and the number of removed characters and giving a high weighting on the number of the replaced characters.

7. The multinational clinical data standardization method of claim 4, wherein the step of performing standardization includes the steps of arranging names of individual entities corresponding to the two sets of character strings, having similarities equal to or higher than a predetermined threshold value, to be one name; and performing standardization of the multinational clinical data by reflecting results from the step of arrangement.

8. The multinational clinical data standardization method of claim 1, further including the step of converting multinational clinical data written in a hierarchical database (DB) format into data in a relational DB format, wherein, in the step of outputting names of individual entities, the names of individual entities are outputted from the multinational clinical data written in the relational DB format by using a neural network model.

9. A multinational clinical data standardization device comprising:
a memory to store multinational clinical data; and
a processor to output names of individual entities from multinational clinical data using a neural network model, to refine the names of individual entities, to calculate similarities of the refined names of the individual entities, and to standardize the multinational clinical data by reflecting results of the similarity calculation.
